(19) United States
(12) Patent Application Publication
(10) Pub. No.: US 2009/0192602 A1
(43) Pub. Date: Jul. 30, 2009

(54) Deformable Sizer and Holder Devices for Minimally Invasive Cardiac Surgery

(75) Inventor: Stephen Kuehn, Woodbury, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/358,820

(22) Filed: Jan. 23, 2009

Related U.S. Application Data
(60) Provisional application No. 61/062,414, filed on Jan. 25, 2008.

(51) Int. Cl. A61F 2/24

(52) U.S. Cl. 623/2.11

(57) ABSTRACT

Described is a device for sizing a heart valve annulus by a minimally invasive route, the device comprising: a sizing plate having first and second major surfaces, wherein the sizing plate comprises a deformable material such that the sizing plate can deform and fit through the minimally invasive route; and an elongate handle to which the sizing plate is attached. Also described is a similarly deformable device for holding an annuloplasty device. Additionally, methods of using the deformable sizing and holding devices are disclosed.

### PRIORITY

The present non-provisional patent application claims benefit from U.S. Provisional Patent Application having Ser. No. 61/062,414, filed on Jan. 25, 2008, by Kuehn et al., and titled SIZER, HOLDER AND DELIVERY DEVICES FOR MINIMALLY INVASIVE ANNULOPLASTY SURGERY, wherein the entirety of said provisional patent application is incorporated herein by reference.

### TECHNICAL FIELD

This invention generally relates to devices and methods of repair and replacement of heart valves. In particular, the invention relates to devices for measuring the size of a heart valve annulus and for holding and delivering an annuloplasty device to the annulus during minimally invasive cardiac surgery.

### BACKGROUND OF THE INVENTION

Heart valve disease is a widespread condition in which one or more of the valves of the heart fails to function properly. Various surgical techniques may be used to replace or repair a diseased or damaged valve. Damaged leaflets of the valve may be excised and the annulus sculpted to receive a replacement valve. Another less drastic method for treating defective valves is repair or reconstruction by annuloplasty, in which the effective size of the valve annulus is contracted and reinforced, by attaching a prosthetic annuloplasty ring or band to an interior wall of the heart around the valve annulus. The annuloplasty ring or band is designed to support the functional changes that occur during the cardiac cycle, while maintaining leaflet coaptation and valve integrity.

To perform successful valve replacement and annuloplasty surgeries, the size of the valve annulus must be accurately measured. Sizing may be achieved by measuring the width and the height of the anterior leaflet of the mitral valve, for example, using sizing obturators. Another way to size the annulus is to use valve sizers, which resemble the shape of the valve annulus and are provided in various sizes. In order to use valve sizers, a surgeon estimates the valve annulus size and selects a sizer accordingly. The sizer is guided into proximity of the annulus with a handle. If the sizer is not judged to be the correct size, it is withdrawn, and replaced by a different sizer. Once the size of the annulus has been determined, a properly sized valve or annuloplasty device may be selected. The selected annuloplasty device is placed on a holder device that is delivered to the annulus. The annuloplasty device is attached to the annulus and removed from the holder device. The delivery device and holder device are then removed from the body.

Surgical techniques for annuloplasty surgery are typically performed open-chest. This usually requires the patient to be placed on a cardiac bypass machine to pump and oxygenate the blood while the surgeon operates on the stopped heart muscle. Open-chest surgery can be very traumatic for the patient and recovery can take many months. Additionally, such surgery may not be an option for some patients due to limited possibility for recovery, concurrent disease, or age.

For these reasons, it is desirable to use minimally invasive cardiac surgical techniques for valve repair. However, these procedures reduce the available space to deliver surgical instruments to a surgical site, and reduce the space in which surgical instruments may be operated within the area of the surgical site. Therefore, such procedures require surgical instruments with appropriate size and maneuverability that accommodate the limited space.

Traditional annuloplasty and valve sizing and holding instruments were designed for use with open-chest surgery that exposes the appropriate regions of the heart to complete and open access through the open chest wall. The ability of these instruments to fit through significantly reduced surgical field access points was not a necessary criteria for their design. Advances in the surgical field toward minimally invasive techniques has created significant new challenges for the design of new instruments and the development of new techniques for using these instruments to successfully complete procedures in limited access surgical fields.

Exemplary types of minimally invasive cardiac surgery include atrio-ventricular valve repair, reconstruction, or replacement surgical procedures. In particular, the replacement of the valves and repair of valve annulus dilation using annuloplasty devices can employ minimally invasive techniques.

Despite the current existence of sizing devices for sizing a valve annulus and holding devices for holding annuloplasty devices, there is still a need for improved devices, and in particular those devices that may be used during minimally invasive cardiac surgical procedures.

### SUMMARY OF THE INVENTION

Embodiments of the present invention include sizer devices, holder devices, and delivery devices that are made, configured and/or may be manipulated to fit through significantly reduced surgical field access points and may be used in reduced surgical fields of operation. In particular, the sizer and holder devices are deformable, such that the devices may deform, bend or flex in order to fit through reduced surgical field access points.

The embodiments of the present invention offer an advantage that they may be used during minimally invasive cardiac surgery to fit through significantly reduced surgical field access points and in reduced surgical fields of operation. In doing so, the embodiments of the present invention reduce the physical trauma to the patient by eliminating the need to perform a complete sternotomy, and reduce the time spent in surgery. The embodiments of the present invention also allow annuloplasty surgery to be performed on patients that would not otherwise be able to have the surgery involving open-chest techniques.

A first aspect of the present invention is a device for sizing a heart valve annulus by a minimally invasive route. One embodiment of the device comprises: a sizing plate having first and second major surfaces, wherein the sizing plate comprises a deformable material such that the sizing plate can deform and fit through the minimally invasive route; and an elongate handle to which the sizing plate is attached. The device may further comprise an attachment hub on the first or second major surface of the sizing plate, wherein the attachment hub releasably attaches the sizing plate to the elongate handle for delivery to the heart valve annulus by the minimally invasive route. The deformable material may comprise a urethane, a silicone, or a shape memory polymer or metal. The sizing plate may include two notches or two markings that correspond to the trigones and/or commissures of the heart valve annulus and are used to size the heart valve annulus. The sizing plate may be generally planar in configuration or may have a three-dimensional configuration. The sizing plate may include an opening through which a suture or other lanyard may be passed in order to tether the device.

A second aspect of the present invention is a device for holding an annuloplasty device for delivery to a heart valve annulus by a minimally invasive route. One embodiment of the device comprises: a holding plate to which the annuloplasty device may be attached, having first and second major surfaces, wherein the holding plate comprises a deformable material such that the holding plate can deform and fit through the minimally invasive route; and an elongate handle to which the holding plate is attached. The device may further comprise an attachment hub on the first or second major surface, wherein the attachment hub releasably attaches the holding plate to the elongate handle for delivery to the heart valve annulus by the minimally invasive route. The deformable material may comprise a urethane, a silicone, or a shape memory polymer or metal. The holding plate may be generally planar in configuration or may have a three-dimensional configuration. The holding plate may include at least one opening through which a suture or other lanyard may be passed in order to tether the device. The device may further comprise the annuloplasty device attached to the holding plate.

A third aspect of the present invention is a method of sizing a heart valve annulus. One embodiment comprises the steps of: receiving a device for sizing a heart valve annulus by a minimally invasive route, the device comprising: a sizing plate having first and second major surfaces, wherein the sizing plate comprises a deformable material such that the sizing plate can deform and fit through the minimally invasive route; and an elongate handle to which the sizing plate is attached; inserting the sizing plate and elongate handle into the minimally invasive route; comparing the sizing plate to the heart valve annulus in order to determine the size of the heart valve annulus; and removing the sizing plate and elongate handle from the minimally invasive route. The device may further comprise an attachment hub on the first or second major surface that releasably attaches the sizing plate to the elongate handle, and the method may further comprise the step of attaching the elongate handle to the sizing plate.

A fourth aspect of the present invention is a method of delivering an annuloplasty device to a heart valve annulus. One embodiment may comprise the steps of: receiving a device for holding the annuloplasty device for delivery to a heart valve annulus by a minimally invasive route, the device comprising: a holding plate to which the annuloplasty device is attached, having first and second major surfaces, wherein the holding plate comprises a deformable material such that the holding plate can deform and fit through the minimally invasive route; and an elongate handle to which the holding plate is attached; inserting the holding plate and annuloplasty device and elongate handle into the minimally invasive route; delivering and attaching the annuloplasty device to the heart valve annulus; removing the annuloplasty device from the holding plate; and removing the holding plate and elongate handle from the minimally invasive route. The device may further comprise an attachment hub on the first or second major surface that releasably attaches the holding plate to the elongate handle, and the method may further comprise the step of attaching the elongate handle to the holding plate.

### BRIEF DESCRIPTION OF THE DRAWINGS

The present invention will be further explained with reference to the appended Figures, wherein like structure is referred to by like numerals throughout the several views, and wherein:

FIG. 1A is a perspective view of a sizer device, in accordance with the present invention, in a planar configuration;

FIG. 1B is a perspective view of the sizer device of FIG. 1A with a portion flexed as indicated by arrow;

FIG. 2A is a perspective view of the sizer device of FIGS. 1A and 1B adjacent a slot; and

FIG. 2B is a perspective view of the sizer device and slot of FIG. 2A showing the sizer device inserted in the slot and in a deformed configuration.

### DETAILED DESCRIPTION OF PREFERRED EMBODIMENTS

Embodiments of the present invention include sizer devices, holder devices, and delivery devices that are made, configured and/or may be manipulated to fit through significantly reduced surgical field access points and may be used in reduced surgical fields of operation. In particular, the sizer and holder devices are deformable, such that the devices may deform, bend or flex in order to fit through reduced surgical field access points. The sizer devices and holder devices are preferably made to be attached and detached from the delivery devices of the present invention. However, the sizer and holder devices may be permanently attached to the delivery devices.

Particularly, the sizer devices, holder devices and delivery devices will be discussed with regard to their use during annuloplasty surgery. During annuloplasty surgery, the purpose of the delivery device is to first deliver a sizer device to a valve annulus that is in need of repair in order to size the annulus, and then after removal of the sizer device from the body and the delivery device, the delivery device is next used to deliver a holder device with an attached annuloplasty device to the valve annulus for implantation of the annuloplasty device. Although the present application addresses annuloplasty surgery, it is contemplated that the present invention or features thereof may be used during other minimally invasive surgical procedures as well.

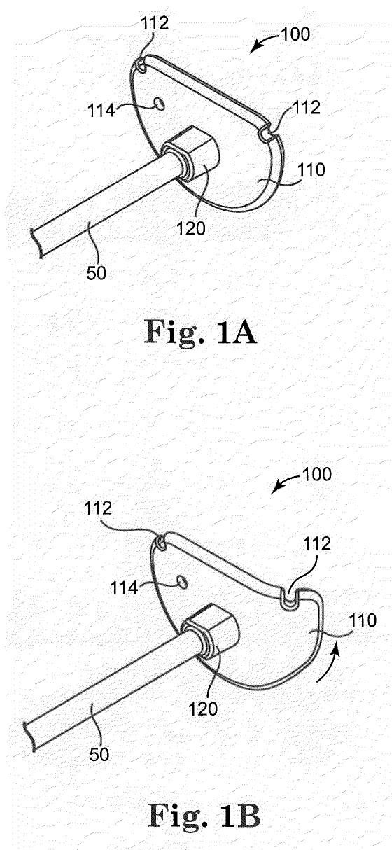

With reference to the accompanying figures, wherein like components are labeled with like numerals throughout the several figures, and, initially, to FIGS. 1A and 1B, one embodiment of the present invention is shown. Sizer device 100 is a device used for sizing a valve annulus for selection of a properly sized corresponding annuloplasty device. The sizer device 100 shown may be compared to an inter-trigonal distance (or inter-commissural distance) of a mitral valve annulus, in particular. The inter-trigonal (or inter-commissural) distance of the mitral valve annulus is preferred to be measured in order to be able to choose an appropriate annuloplasty device for repair of the mitral valve. The sizer device 100 of the present invention will correspond to one of a plurality of possible sizes of annuli, i.e. inter-trigonal or inter-commissural distances. Although the sizer device 100 applies to the mitral valve, it is also contemplated that the present invention may also apply to other valves, such as the tricuspid valve.

The sizer device 100 comprises a sizing plate 110 and an attachment hub 120. The attachment hub 120 is preferably located on one of two major surfaces of sizing plate 110. The attachment hub 120 provides a place for an elongate handle 50 to connect to the sizer device 100. The elongate handle 50 may be permanently or releasably attached to the sizer device 100. It is contemplated that the sizer device 100 may be snap-fit onto the elongate handle 50, threaded onto the elongate handle 50, or magnetically bound to the elongate handle 50, for example. Alternatively, the attachment hub 120 could serve as a protrusion that can be grasped by a long handled surgical instrument, or attached to the handle 50 by any other suitable attachment means.

The purpose of the elongate handle 50 is to deliver the sizer device 100 adjacent or near the valve annulus being sized. The surgeon performing the sizing may hold the elongate handle 50 from outside of a patient's body.

The elongate handle 50 may comprise a metal wire. However, the present invention is not limited to the use of metal wire for the elongate handle 50, and other materials are also contemplated, such as polymers, for example.

A plurality of different sizes of sizer devices 100 are possible. The size of each sizer device 100 is based upon the length of the major axis of the device, which corresponds to an inter-trigonal (or inter-commissural) distance of an annulus. Annuloplasty devices are generally available starting with a major axis length of 24 mm, and increasing by 2 mm each up to about 40 mm. Therefore, sizer devices should be capable of being provided with all of the sizes that correspond to the sizes of the annuloplasty devices.

The sizing plate 110 of the sizer device 100 is D-shaped in the figures, however, the sizing plate 110 may have one of a plurality of possible two-dimensional (2D) and/or three-dimensional (3D) shapes. The shape of the sizing plate 110 depends upon, e.g., the type of valve being sized, the disease state of the valve, the shape of a corresponding annuloplasty device, etc.

The sizing plate 110 is shown having a continuous surface. However, the sizing plate 110 may alternatively be discontinuous and may include voids, opening or through holes (e.g., hole 114) that may be useful, for example, in tying a suture loop or other type of lanyard through the device to act as a tether should the sizer device 100 become disconnected from the handle 50. Alternatively, the sizing plate 110 could include other means (e.g., hooks or loops) that could allow a suture or other type of lanyard to loop through or be attached to the plate 110 to act as a tether.

The sizing plate 120 is preferably made from biocompatible material that is also flexible or deformable. The material could have a degree of deformability to minimize tissue trauma while introducing the sizer device 100 through a reduced surgical access site (e.g., transthoracic site). The flexible material is also preferably optically transparent, but could also be opaque. Some particular materials that may be used include a number of polymers and elastomers, including, but not limited to, urethanes and silicones. If a polymer is used, the polymer may be compounded with a ferrous metal to achieve magnetic properties or any other radiopaque material for identification under x-ray. Also, the material could comprise a shape memory polymer or metal, such as Nitinol™, for example. Other similar materials having such similar properties are also contemplated by the present invention. The material is preferably able to regain its predefined shape after being deformed in some way.

Sizer device 100 preferably includes a thin layer of the flexible material. The thickness is minimized in order to preferably prevent optical distortion through the sizing plate 110 and/or in order to allow the sizing plate 110 to deform and fit through relatively small openings.

FIG. 1A shows the sizer device 100 with the sizing plate 110 in a planar configuration, and FIG. 1B shows the sizing plate 110 in a flexed, bent or deformed configuration, as indicated by the arrow. It is contemplated, however, that the sizing plate 110 may be flexed, bent or deformed in a plurality of different directions, and such directions are not limited to those shown in the figures.

Sizer device 100 may comprise a material that allows the sizer device 100 to be made using injection molding techniques. Using injection molding techniques to make such devices is an advantage, in that it saves time and effort in production. Also, compounding metallic materials with the injection molding polymer can give the elastomeric material magnetic or radiopaque properties.

Sizing plate 120 preferably includes marks, such as cut-out segments, notches or recesses 112, that may be used as left and right trigone (or commissure) position identifiers. Other types of marks or markings include printing, and/or laser engraving, etc.

When using the sizer device 100 to size a valve annulus, the surgeon inserts the sizer device 100 adjacent a valve annulus and, first, checks the inter-trigonal distance. The user may check the distance by determining if the notches 112 line up with the left and right trigones of the annulus. The inter-trigonal distance generally determines the size of the valve annulus. Alternatively, the commissures on the annulus are used to determine the size, which is called the inter-commissural distance. The two valve commissures (posterior and anterior) define a distinct region where the anterior and posterior leaflets of the mitral valve come together at their insertion into the annulus, and may alternatively be used to determine the size of the annulus.

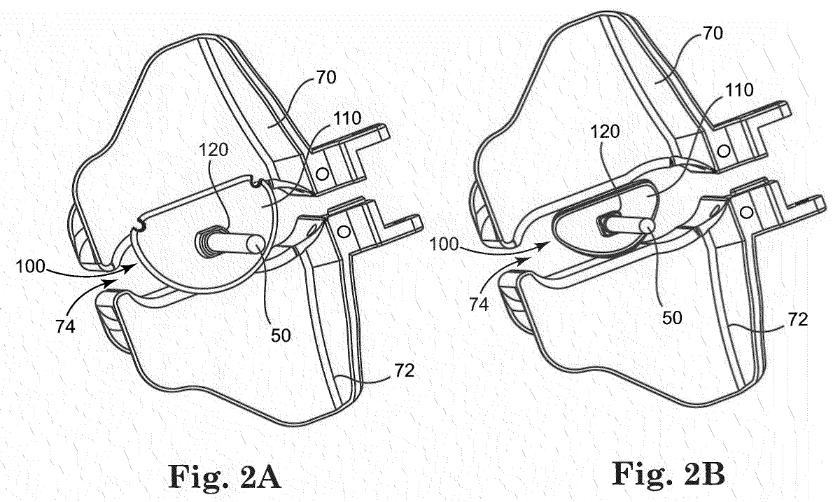

FIGS. 2A and 2B show a first 70 and second 72 slot-forming segments that are held a certain distance apart in order to form a slot 74 of a given size. Preferably, the size of slot 74 corresponds to a size of surgical access site in a minimally invasive cardiac surgical procedure, such as annuloplasty surgery (e.g., a transthoracic site that is between two ribs). FIG. 2A shows sizer device 100 adjacent slot 74. FIG. 2B shows sizer device 100 being inserted in the slot 74. The sizing plate 110 is deformed in order for the sizer device 100 to fit through the slot 74. FIG. 2B demonstrates the usefulness of sizer device 100, particularly during minimally invasive cardiac surgery.

Another embodiment of the present invention is a deformable holder device. The description above also generally applies to such a holder device. Instead of being used to measure the size of a valve annulus, however, the holder device holds an annuloplasty device for delivery during annuloplasty surgery. These holder devices include some means for attaching an annuloplasty device to a holding plate portion. For example, the holder plate may include suture holes through which sutures or other types of lanyards may secure an annuloplasty device to the holder device. The deformability of the holder device assists in allowing an annuloplasty device to be delivered through a reduced surgical access site, and also allows the holder device to be removed easily through the reduced surgical access site. The holder device may be integrated with an elongate handle. Therefore, the present invention also includes deformable holder devices.

Additionally, the present invention includes any delivery device that incorporates either a deformable sizer device or deformable holder device of the present invention. Further, the present invention also includes methods of making and using the sizer, holder and delivery devices of the present invention.

It is to be understood that while particular embodiments of the invention have been illustrated for use in typical valve repair procedures, various modifications to shape, and arrangement of parts can be made as may be desirable for varying applications as may relate to valve sizes or later developed techniques. The invention should not be considered limited to the specific methods and devices precisely described herein. On the contrary, various modifications will be apparent to those of ordinary skill upon reading the disclosure. Although certain embodiments are described with reference to the mitral valve, use with other valves or anatomical structures is also contemplated. The foregoing detailed description has been given for clarity of understanding only. No unnecessary limitations are to be understood there from. The entire disclosure of any article, patent or patent application identified herein is hereby incorporated by reference.

1. A device for sizing a heart valve annulus by a minimally invasive route, the device comprising:
a sizing plate having first and second major surfaces, wherein the sizing plate comprises a deformable material such that the sizing plate can deform and fit through the minimally invasive route; and
an elongate handle to which the sizing plate is attached.

2. The device of claim 1, further comprising an attachment hub on the first or second major surface of the sizing plate, wherein the attachment hub releasably attaches the sizing plate to the elongate handle for delivery to the heart valve annulus by the minimally invasive route.

3. The device of claim 1, wherein the deformable material comprises a urethane or a silicone.

4. The device of claim 1, wherein the deformable material comprises a shape memory polymer or metal.

5. The device of claim 1, wherein the sizing plate includes two notches that correspond to the trigones and/or commissures of the heart valve annulus and are used to size the heart valve annulus.

6. The device of claim 1, wherein the sizing plate includes two markings that correspond to the trigones and/or commissures of the heart valve annulus and are used to size the heart valve annulus.

7. The device of claim 1, wherein the sizing plate is generally planar in configuration.

8. The device of claim 1, wherein the sizing plate has a three-dimensional configuration.

9. The device of claim 1, wherein the sizing plate includes an opening through which a suture or other lanyard may be passed in order to tether the device.

10. A device for holding an annuloplasty device for delivery to a heart valve annulus by a minimally invasive route, the device comprising:
a holding plate to which the annuloplasty device may be attached, having first and second major surfaces, wherein the holding plate comprises a deformable material such that the holding plate can deform and fit through the minimally invasive route; and
an elongate handle to which the holding plate is attached.

11. The device of claim 10, further comprising an attachment hub on the first or second major surface, wherein the attachment hub releasably attaches the holding plate to the elongate handle for delivery to the heart valve annulus by the minimally invasive route.

12. The device of claim 10, wherein the deformable material comprises a urethane or a silicone.

13. The device of claim 10, wherein the deformable material comprises a shape memory polymer or metal.

14. The device of claim 10, wherein the holding plate is generally planar in configuration.

15. The device of claim 10, wherein the holding plate has a three-dimensional configuration.

16. The device of claim 10, wherein the holding plate includes at least one opening through which a suture or other lanyard may be passed in order to tether the device.

17. The device of claim 10, further comprising the annuloplasty device attached to the holding plate.

18. A method of sizing a heart valve annulus, the method comprising the steps of:
receiving a device for sizing a heart valve annulus by a minimally invasive route, the device comprising:
a sizing plate having first and second major surfaces, wherein the sizing plate comprises a deformable material such that the sizing plate can deform and fit through the minimally invasive route; and
an elongate handle to which the sizing plate is attached;
inserting the sizing plate and elongate handle into the minimally invasive route;
comparing the sizing plate to the heart valve annulus in order to determine the size of the heart valve annulus; and
removing the sizing plate and elongate handle from the minimally invasive route.

19. The method of claim 18, wherein the device further comprises an attachment hub on the first or second major surface that releasably attaches the sizing plate to the elongate handle, and the method further comprises the step of attaching the elongate handle to the sizing plate.

20. A method of delivering an annuloplasty device to a heart valve annulus, the method comprising the steps of:
receiving a device for holding the annuloplasty device for delivery to a heart valve annulus by a minimally invasive route, the device comprising:
a holding plate to which the annuloplasty device is attached, having first and second major surfaces, wherein the holding plate comprises a deformable material such that the holding plate can deform and fit through the minimally invasive route; and
an elongate handle to which the holding plate is attached;
inserting the holding plate and annuloplasty device and elongate handle into the minimally invasive route;
delivering and attaching the annuloplasty device to the heart valve annulus;
removing the annuloplasty device from the holding plate; and
removing the holding plate and elongate handle from the minimally invasive route.

21. The method of claim 20, wherein the device further comprises an attachment hub on the first or second major surface that releasably attaches the holding plate to the elongate handle, and the method further comprises the step of attaching the elongate handle to the holding plate.
